(19) United States
(12) Patent Application Publication
(10) Pub. No.: US 2006/0293761 A1
(43) Pub. Date: Dec. 28, 2006

(54) Hydraulic knee prosthesis

(75) Inventors: Hans-Uwe Baumann, Stuttgart (DE); Andreas Kluge, Renningen (DE); Michael Kaschuba, Renningen (DE)

Correspondence Address: CROWELL & MORING LLP;INTELLECTUAL PROPERTY GROUP, P.O. BOX 14300, WASHINGTON, DC, 20044-4300, US

(73) Assignee: Dr. Ing. h.c.F. Porsche Aktiengesellschaft, Stuttgart (DE)

(21) Appl. No.: 11/471,484

(22) Filed: Jun. 21, 2006

(30) Foreign Application Priority Data
Jun. 23, 2005 (DE) 10 2005 029 160.0

(51) Int. Cl. A61F 2/74

(52) U.S. Cl. 623/26; 623/44

(57) ABSTRACT

A hydraulic knee prosthesis for a leg prosthesis includes a joint unit, a foot connection unit, and a rod connecting these components. The joint unit includes a rotatory pin with a damper chamber and a damper wing screw movable therein under the control of a central hydraulic control unit.

This application claims the priority of German application 10 2005 029 160.0, filed Jun. 23, 2005, the disclosure of which is expressly incorporated by reference herein.

The present invention relates to a knee joint prosthesis, as used in a leg prosthesis for adaptation to a thigh stump, as is already disclosed in German document DE 195 06 426 C1.

According to this known arrangement, a so-called brake-action knee joint includes an upper prosthetic part, a lower prosthetic part, a pin connected in a torsionally rigid fashion to a joint section, and a rocker which forms a central part of the joint. The rocker has an extension-side end, fixed to a rocker pin lying parallel, ventrally, and distally with respect to the joint pin, and a flexion-side end. The rocker surrounds the joint pin, and a braking device is activated by foot loading. The joint pin is arranged in a displacer chamber filled with hydraulic oil, with this displacer chamber divided into an extension chamber and a flexion chamber that are mutually connected by way of an oil line, which can be completely or partially closed by a valve plunger.

One object of this invention is to constructively improve the hydraulic knee joint function for leg prostheses, and to bring the knee joint function selectability still closer to the natural range of motion when ambulating, walking, climbing stairs, cycling, or performing other, different movements.

In one embodiment of the present invention, a purely hydraulic knee joint prosthesis is created for thigh amputees covering the normal spheres of the wearer in the best way possible. This embodiment is applicable to domestic, occupational, leisure and sporting activities, such as hiking or cycling. Functions such as free mobility of the unloaded joint, brake stabilization in the standing position, walking at variable speeds, support of extension initiation—i.e. onset of the swing phase, and definable time dependent resistance to drop off when climbing and descending stairs, as well as ascending or descending ramps and sitting down and standing up and kneeling, are possible.

### BRIEF DESCRIPTION OF THE DRAWING

An embodiment of the hydraulic knee prosthesis is illustrated in the sole drawing FIGURE and will be explained in more detail below.

### DETAILED DESCRIPTION OF THE INVENTION

The knee joint prosthesis is made up of a joint unit G, a foot connection unit F, as well as a rod R that connects the units. Joint unit G includes a rotational pin 10. A rotation damper actuates the joint unit, and the joint unit has a complex hydraulic control for standing up brake action, control, and timed dependent actuation, which will be explained in more detail hereinafter. The knee joint prosthesis further comprises a connection adapter AS for thigh stump connection, and a foot connection adapter AF to connect the foot prosthesis.

The rotational pin 10 of the knee joint prosthesis is located at the natural height of the joint pin. The knee pin 10 itself is enclosed in a hydraulically filled damper chamber 13. The joint pin is located in an eye-shaped pin box 11, which comprises a wing screw 12 that moves according to the motion of the knee joint in the damper chamber 13 filled with hydraulic oil. This interaction provides for the brake-action in the knee joint. The different modes of motion are adjusted by a hydraulic control, which controls the motion of rotational pin 10 in the eye-shaped pin box 11.

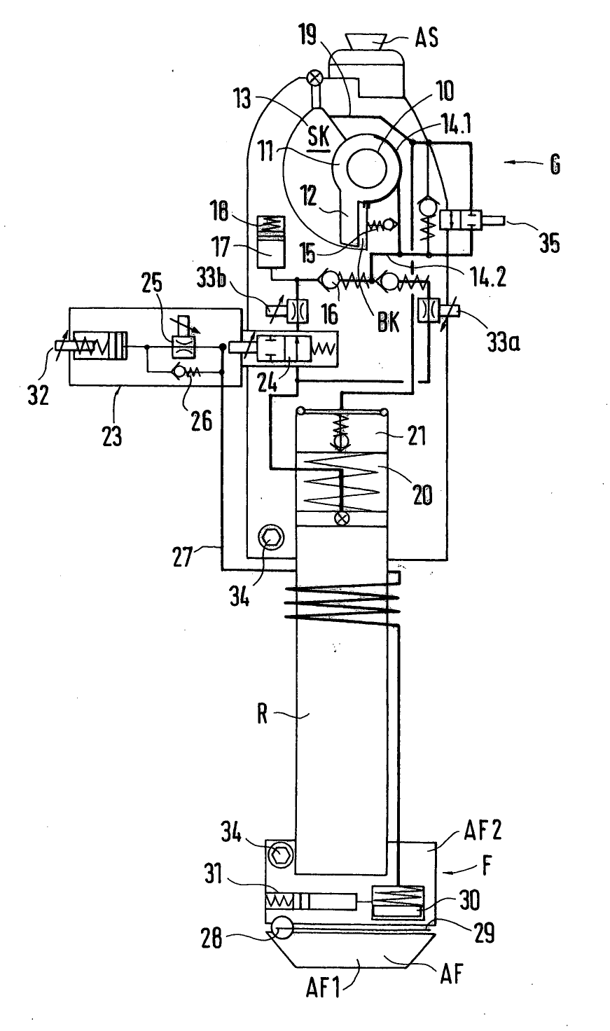

During motion of the knee joint, the individual sections or chambers change in the damper chamber 13. As illustrated in FIG. 1, with an extended leg, an extension chamber SK has maximum length and volume, and a flexion chamber BK has minimum length and volume. Upon rotation of the wing screw 12 during flexion of the knee joint, the lengths of the extension chamber SK and the flexion chamber BK change in such a way that, at maximum flexion, the extension chamber SK has a minimal size and the flexion chamber BK occupies the maximum volume of the damper chamber.

During flexion of the knee joint from the extended end-position, hydraulic fluid flows through an oil control line that is arranged in the pin box, and ensures oil inflow in and outflow of the damper chamber 13. This oil control line can be divided into a first section 14.1 and a second section 14.2. The adjustment of the oil flow through the control line 14 is provided by the shape and contours of the pin box and the resulting transversal openings in the angular position. A check valve 15 is disposed between sections 14.1 and 14.2 of the control line 14, which valve leads back to flexion chamber BK in the damper chamber 13. This check valve 15 bypasses a closed hydraulic control line in order to initiate the opposite movement of the knee back to the extension position without resistance. During knee flexion and movement of the wing screw 12 within the damper chamber 13, the hydraulic oil is displaced from the extension chamber SK through the oil lines 14.1 and 14.2 into the flexion chamber, and parallel to this through a check valve 16 into a balancing chamber 17. The balancing chamber 17 comprises a spring-loaded plunger 18, whereby oil fluctuations due to volume changes can be balanced by means of the temperature. The balancing chamber 17 further balances oil loss due to leaks and together with the spring-loaded plunger 18 ensures oil supply to support overpressure situations in order to avoid air penetration into the complete system.

The function of the hydraulic knee joint has been described above. In summary, it should again be mentioned that the hydraulic brake-action knee joint is essentially implemented by the rotational pin, the eye-shaped pin box 11 with the damper wing screw, the damper chamber 13 and the hydraulic control line, which is arranged in the pin box and displaces oil from the extension chamber SK into the flexion chamber BK by adjusting the wing screw.

The following actuators ensure the different modes of function of the hydraulic knee joint.

After flexion of the knee joint, extension in a simple fashion should be ensured. For this purpose, the extension chamber SK is connected by a further hydraulic line 19 to a hydraulic spring-loaded accumulator 20, which is actuated by the circulating oil of the damper chamber 13 in order to release the energy generated during flexion for motion reversal. The spring-loaded accumulator 20 comprises a plunger 21, which is actuated by oil on both sides and is located centrally between the oil spaces in front and behind the damper wing screw 12, i.e. between the extension chamber and the flexion chamber. The plunger 21 further includes an integrated check valve to ensure that at extension the initial position can always be reached, even in case of oil leaks.

A switching valve 35 integrated into the hydraulic line 19 can be accessed by the wearer from the outside, and allows switching from the walking to the cycling function.

The entire hydraulic area is controlled by a central valve unit 23. This central valve unit 23 consists of a central valve 24 with adjustable throttle 25, a check valve 26 arranged parallel to the throttle 25, and a control line 27 located between the central valve 24 and the parallel connection consisting of the adjustable throttle 25 and the check valve 26, which line is directly connected to the foot connection adapter AF and actuated by it.

The foot connection unit F with the foot connection adapter AF consists of a two-part assembly. Both parts AF1 and AF2 of the assembly are mounted kinematically in relation to one another, and the bearing shaft is identified with reference numeral 28. Additionally, an elastomer insert 29 is disposed between both parts of the foot connection adapter AF. According to the way the foot is put down, a deflecting angle is generated between both parts of the foot adapter, which causes an offset motion in a pressure plunger 30 to trigger a control signal via the control line 27. The following features should be noted.

The foot connection unit is configured such that lowering the foot near-ground is recognized, and a hydraulic signal is released by the control line to close the damper chamber 13.

The rolling-off phase of the foot is recognized by the hydraulic control system, which opens the damper chamber 13.

Kinematics, positioning and the design of the foot connection unit F, as well as the hydraulic transmission conditions of the complete control circuit are designed in the embodiment of the invention such that the required activation paths and/or angles at the foot connection unit F are kept to a minimum. The positioning and connection of the foot connection unit F by means of elastomer or vulcanized rubber inserts 29 allow both parts AF1 and AF2 of the foot connection unit to be positioned relative to one another and separated acoustically. In addition, by means of elastomer insert 29, a defined activation path and/or angle relative to one another is possible, which can be reversed load free by elasticity to the initial position. In the foot connection unit F, a balancing chamber with plunger is further provided for taking up volume oscillations of the hydraulic oil due to volume fluctuations caused by temperature changes. This balancing chamber is identified with reference numeral 31 in the FIGURE. The entire range of motion is controlled by the central valve unit 23. This central valve unit 23 is a time dependent mechanism, wherein, following a trigger signal, for example shortly after putting down the foot, the damper circuits are again opened partially or completely in order to rapidly obtain a high and subsequently low damping effect. Thereby treading is maximally ensured for a short period of time, and a defined partial mobility is immediately allowed, as required with stairs, when upon stepping on the step and subsequent pressing down the joint reaching the next step has to be ensured. This signal detected on the foot by the connection adapter which actuates the plunger 30, is conducted via the control line 27 to the central valve unit 23.

The central valve unit 23 further comprises an adjustable path limiter 32, which can be adjusted manually by the person concerned. This path limiter allows exact adjustment of the drop off resistance of the knee joint prosthesis to the wearer's requirements.

Two additional adjustable regulators are identified with reference numerals 33a and 33b and allow separate adjustment of flexion and extension, as it directly acts on the oil flow, which flows from the damper chamber 13 at motion.

The rod R connecting the joint and foot parts is respectively connected to the joint unit G and foot connection unit AF by means of a clamp 34. The length of the rod allows exact adjustment of the height of the knee pin 10 to the height of the wearer.

The individual components are surface-treated, and the overall strength was improved by hard anodizing. The surface-treatment of all affected components, such as the hydraulic block, the damper wing screw, the valve components, and the Teflon-lined plunger optimizes the sliding and abrasion properties. Moreover, the valve parts are made of lightweight metal to minimize weight.

The knee joint prosthesis described above, and the hydraulic actuator of the knee prosthesis, optimally cover every motion requirement of the wearer, and have manifold applications in everyday life.

As a result of its configuration, the complete system has a spring-loaded accumulator integrated in the oil circuit, whereby the energy generated by the system can again be released, and thus be preserved for the complete system.

The foregoing disclosure has been set forth merely to illustrate the invention and is not intended to be limiting. Since modifications of the disclosed embodiments incorporating the spirit and substance of the invention may occur to persons skilled in the art, the invention should be construed to include everything within the scope of the appended claims and equivalents thereof.

We claim:

1. A hydraulic prosthesis comprising:
a joint unit,
a foot connection unit,
a rod connecting the joint unit and the foot connection unit, and
a hydraulic control device,
wherein the joint unit includes a rotational pin, a damper chamber, and a damper wing screw movable therein under control of the hydraulic control device.

2. The hydraulic prosthesis as claimed in claim 1, wherein the hydraulic control device includes a central valve unit, wherein the foot connection unit is connected to the central valve unit via a control line, and wherein deflection between parts of the foot connection unit provides a control signal via the control line to control the central valve unit.

3. The hydraulic prosthesis as claimed in claim 2, wherein the central valve unit controls damper and brake actions of the damper chamber by actuating different check valves.

4. The hydraulic prosthesis as claimed in claim 1, further comprising adjustable regulators and adjustable path limiters which allow manual control of damper properties of the prosthesis.

5. The hydraulic prosthesis as claimed in claim 2, further comprising adjustable regulators and adjustable path limiters which allow manual control of damper properties of the prosthesis.

6. The hydraulic prosthesis as claimed in claim 3, further comprising adjustable regulators and adjustable path limiters which allow manual control of damper properties of the prosthesis.

7. The hydraulic prosthesis as claimed in claim 1, further comprising a mechanism adapted to compensate for oil loss.

8. The hydraulic prosthesis as claimed in claim 7, wherein the mechanism ensures oil supply to support overpressure situations.

9. The hydraulic prosthesis as claimed in claim 8, wherein the mechanism includes a balancing chamber and a plunger therein.

10. The hydraulic prosthesis as claimed in claim 2, wherein the deflection is angular deflection.

11. The hydraulic prosthesis as claimed in claim 5, wherein the deflection is angular deflection.

12. The hydraulic prosthesis as claimed in claim 1, wherein the damper wing screw separates the damper chamber into a pair of variable volume chambers.

13. The hydraulic prosthesis as claimed in claim 12, wherein at least one of the variable volume chambers is connected to an accumulator facilitating joint movement.

14. The hydraulic prosthesis as claimed in claim 1, wherein the joint unit is a knee joint unit.

15. The hydraulic prosthesis as claimed in claim 2, wherein the damper wing screw separates the damper chamber into a pair of variable volume chambers.

16. The hydraulic prosthesis as claimed in claim 15, wherein at least one of the variable volume chambers is connected to an accumulator facilitating joint movement.

17. The hydraulic prosthesis as claimed in claim 3, wherein the damper wing screw separates the damper chamber into a pair of variable volume chambers.

18. The hydraulic prosthesis as claimed in claim 17, wherein at least one of the variable volume chambers is connected to an accumulator facilitating joint movement.

19. The hydraulic prosthesis as claimed in claim 4, wherein the damper wing screw separates the damper chamber into a pair of variable volume chambers.

20. The hydraulic prosthesis as claimed in claim 19, wherein at least one of the variable volume chambers is connected to an accumulator facilitating joint movement.
